(19) United States
(12) Patent Application Publication
(10) Pub. No.: US 2010/0144763 A1
(43) Pub. Date: Jun. 10, 2010

(54) Pharmaceutical composition for inhibiting topoisomerase I and method for exploiting drug

(75) Inventors: Chun-Mao Lin, Kaohsiung County (TW); Chwen-Ming Shih, Sijhih City (TW); Jui-Yu Wu, Kaohsiung City (TW)

Correspondence Address: HUDAK, SHUNK & FARINE, CO., L.P.A., 2020 FRONT STREET, SUITE 307, CUYAHOGA FALLS, OH, 44221, US

(73) Assignee: TAIPEI MEDICAL UNIVERSITY, Taipei City (TW)

(21) Appl. No.: 12/592,965

(22) Filed: Dec. 4, 2009

(30) Foreign Application Priority Data
Dec. 5, 2008 (TW) 097147505

(51) Int. Cl. A61K 31/519; A61P 35/00

(52) U.S. Cl. 514/257

(57) ABSTRACT

The invention relates to the compounds isolated from Evodia rutaecarpa (Juss.), in that has been demonstrated to inhibit topoisomerase I. Evodiamine, an alkaloidal compound is reported the topoisomerase I inhibitory activity. The effect of evodiamine acts by stabilizing the covalent complex between topoisomerase I and DNA, which results in a blockade of DNA replication and transcription.

### BACKGROUND OF THE INVENTION

1. Field of the Invention

The present invention relates to a pharmaceutical composition for inhibiting topoisomerase I and a method for exploiting a drug, and particularly to a pharmaceutical composition capable of inhibiting topoisomerase I, and thus applicable to a chemical therapy by a blockade of DNA replication or transcription, and applied to new drug exploitation with the design of the combination of the compound and topoisomerase I (TopoI).

2. Description of Related Art

DNA topoisomerase enzymes regulate the topological state of DNA that is crucial for initiation and elongation during DNA synthesis. Topoisomerase I (TopoI) produces a single strand break in DNA allowing the relaxation of DNA during its replication. The single strand break is then religated, thus restoring DNA double strands. An enzymatic mechanism comprises two sequential transesterification reactions (Teicher, 2008). In a cleavage reaction, an active site tyrosine, such as Tyr 723 in human TopoI, acts as a nucleophile. A phenolic oxygen of the tyrosine attacks a DNA phosphodiester bond, forming an intermediate in which the 3′ end of the broken strand is covalently attached by an O⁴-phosphodiester bond to the tyrosine in the TopoI. A religation step consists of transesterification involving a nucleophilic attack on the intermediate by the hydroxyl oxygen at the 5′ end of the broken strand. The cleavage reaction and the relegation reaction are consistent and reversible. The inhibition of the TopoI transesterification results in a blockade of DNA replication or transcription, thus standing a TopoI-DNA complex intermediate state.

Some TopoI- and TopoII-targeted drugs are reported to stabilize the covalent topoisomerase-DNA complex, thereby preventing the relegation reaction (Liu, 1989). The TopoI reaction intermediate consists of the enzyme covalently linked to a nicked DNA molecule, known as a “cleavable complex”. Covalently bound TopoI-DNA complexes can be trapped and purified because the enzymatic religation is no longer functional. Topoisomerase inhibitors have been developed for antitumor (Feun & Savaraj, 2008; Wethington, Wright & Herzog, 2008), antiviral (Sadaie, Mayner & Doniger, 2004), antibacterial (Anderson & Osheroff, 2001), anti-epileptic (Song, Parker, Hormozi & Tanouye, 2008), and immunomodulation (Verdrengh & Tarkowski, 2003) applications. Camptothecin (CPT) is a representative drug that targets DNA TopoI through trapping a covalent intermediate between TopoI and DNA. However, there are several problems with CPT-derived anticancer agents despite their clinical success, including multidrug resistance (MDR). MDR results from their intracellular concentration being greatly reduced by efflux pumps in a wide variety of tissues which is conferred by P-glycoprotein overexpression (Chu, Kato & Sugiyama, 1997). Accordingly, the clinical application of CPT has a limitation due to easily inducing chemical drug resistance.

Evodiamine (EVO) is an alkaloidal compound isolated from Evodia rutaecarpa (Juss.), and its structural formula as follows.

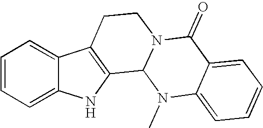

EVO has been reported to possess many physiological functions including vasorelaxation, antiobesity (Wang, Wang, Kontani, Kobayashi, Sato et al., 2008), anticancer (Ogasawara, Matsunaga, Takahashi, Saiki & Suzuki, 2002), and anti-inflammatory (Ko, Wang, Liou, Chen, Chen et al., 2007) effects. Evidence showed that EVO induced apoptosis through an accumulation of the cell cycle at the G2/M phase and initiation of apoptosis (Kan, Huang, Lin & Wang, 2004). The EVO derivatives comprising rutaecarpine, evodiamide, and dehydroevodiamine are usually found in the contents of preparations (Zhou et al. 2006). The structural formulas of rutaecarpine (left structure) and dehydroevodiamine (right structure) are respectively as follows.

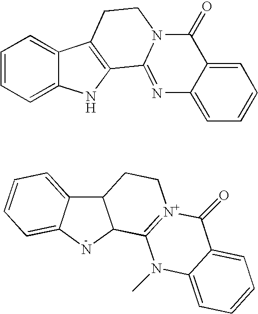

In all bioactivity assays, there are no reports to disclose that EVO and its derivatives act directly on TopoI. The disclosure of the present invention will confirm the functional model of the EVO and its derivatives, and may hence provide beneficial effects of EVO and derivatives thereof to develop a variety of therapeutic applications.

### SUMMARY OF THE INVENTION

The present invention discloses evodiamine (EVO) and its derivatives, rutaecarpine and dehydroevodiamine, that are applicable to stabilize covalent complexes between topoisomerase I (TopoI) and DNA, thereby providing beneficial effects of EVO and derivatives thereof to develop a variety of therapeutic applications.

EVO, an alkaloidal compound isolated from Evodia rutaecarpa (Juss.), can affect many physiological functions. Topoisomerase inhibitors have been developed in a variety of clinical applications. In the present invention, the TopoI inhibitory activity of EVO which may have properties that lead to improved therapeutic benefits is disclosed. EVO is able to inhibit supercoiled plasmid DNA relaxation catalyzed by TopoI. Free-form TopoI is depleted in a time-dependent manner and a dose-dependent manner with EVO treatments in MCF-7 breast cancer cells due to TopoI being fixed on DNA. Moreover, a KCl-SDS precipitation assay is performed to measure the extent of TopoI-trapped chromosomal DNA. The KCl-SDS precipitation method is normally able to precipitate proteins, not precipitate DNA. If DNA is precipitated by the KCl-SDS precipitation, it only occurs in a protein-DNA covalent binding state.

The present invention discloses that the ability of EVO to cause the formation of a TopoI-DNA complex increases in a dose-dependent manner. The results suggest that EVO inhibits TopoI by stabilizing the covalent TopoI-DNA complex.

Referring to FIG. 1, TopoI produces a single strand break in DNA allowing the relaxation of DNA during its replication. The single strand break is then religated, thus restoring DNA double strands (FIG. 1A). After adding EVO or its derivatives to hence stabilize the covalent complex between TopoI and DNA, topoisomerase transesterification is inhibited to result in a blockade of DNA replication or transcription, thus standing a TopoI-DNA complex intermediate state (FIG. 1B) and further inhibiting a cell cycle.

EVO can naturally produce derivatives comprising rutaecarpine and dehydroevodiamine by way of illuminating or heating, and thereby EVO derivatives can be obtained in a common preparation process. Clinical and related prior reports have indicated that topoisomerase inhibitors are applicable to antitumor, antiviral, antibacterial, anti-epileptic, and immunomodulation applications. The present invention firstly demonstrates that EVO has an inhibitory activity of TopoI, and hence it is anticipated that EVO is applicable to antitumor, antiviral, antibacterial, anti-epileptic and immunomodulation applications. Compared to the prior art that ever discloses EVO and its derivative having anticancer, immunomodulation and etc. effects, the antiviral, EVO and its derivative having antibacterial and anti-epileptic effects are never disclosed before.

According to one aspect of the present invention, a pharmaceutical composition for inhibiting TopoI is provided, and the pharmaceutical composition comprises evodiamine or its derivatives. The derivatives further comprises one of evodiamine, rutaecarpine and dehydroevodiamine in combination of a pharmaceutically acceptable salt, solvate, or physiological function derivative thereof. The pharmaceutical composition may inhibit a proliferating cell having TopoI activity. The proliferating cell may comprise a eukaryotic cell, virus or bacterium, and TopoI may be prepared from a virus, eukaryotic cell or recombinant DNA expression in a host.

With these and other objects, advantages, and features of the invention that may become hereinafter apparent, the nature of the invention may be more clearly understood by reference to the detailed description of the invention, the embodiments and to the several drawings herein.

### BRIEF DESCRIPTION OF THE DRAWINGS

The exemplary embodiments of the present invention will be understood more fully from the detailed description given below and from the accompanying drawings of various embodiments of the invention, which, however, should not be taken to limit the invention to the specific embodiments, but are for explanation and understanding only.

FIGS. 1A and 1B illustrate an mechanism of EVO inhibiting TopoI by stabilizing covalent TopoI-DNA complex in accordance with the present invention;

FIG. 2 illustrates an effect of EVO inhibiting supercoiled pcDNA3 plasmid relaxation catalyzed by TopoI from a vaccinia virus in accordance with the present invention;

FIG. 3A illustrates TopoI depletion by EVO in MCF-7 cells treated with EVO (10 μM) for 0˜120 minutes;

FIG. 3B illustrates TopoI depletion by EVO in MCF-7 cells treated with various concentrations of EVO (0˜10 μM); and

FIG. 4 illustrates TopoI-DNA complex trapping activity of EVO in ³H-Thymidine-labeled MCF-7 cells treated with various concentrations (0˜30 μM) of EVO for 60 minutes.

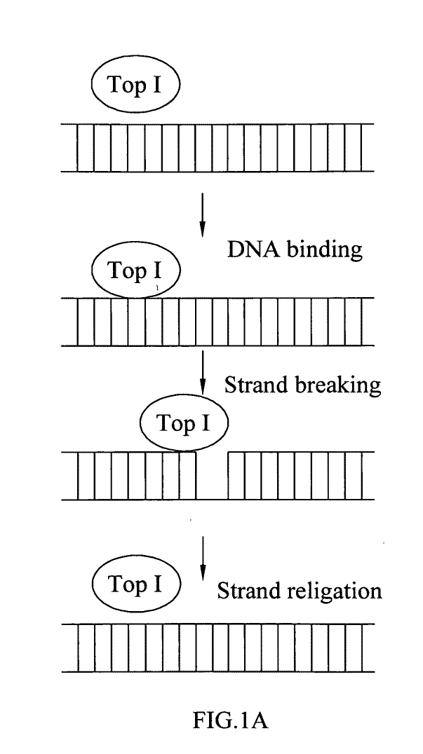

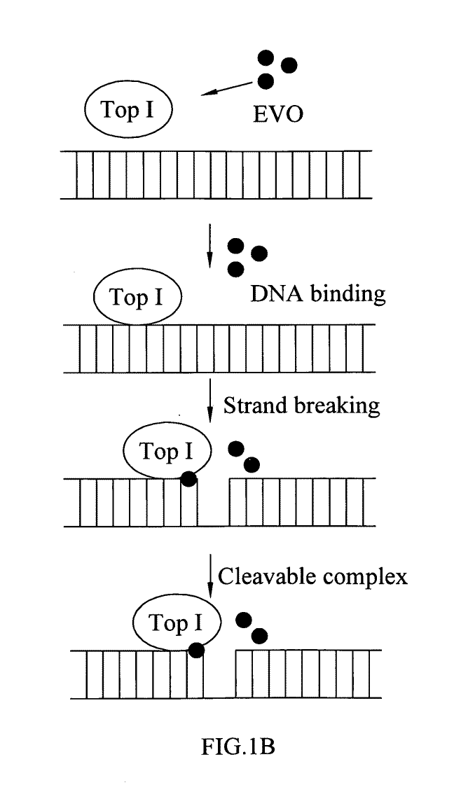

### REFERENCE

Anderson, V. E., Osheroff, N. (2001). Type II topoisomerases as targets for quinolone antibacterials: turning Dr. Jekyll into Mr. Hyde. Curr Pharm Des., 7, 337-353.
Chang, Y. C., Lee, F. W, Chen, C. S., Huang, S. T., Tsai, S. H., Huang, S. H., Lin C. M. (2007). Structure-activity relationship of C6-C3 phenylpropanoids on xanthine oxidase-inhibiting and free radical-scavenging activities. Free Rad. Biol. Med., 43, 1541-1551.
Chu, X. Y., Kato, Y., & Sugiyama, Y. (1997). Multiplicity of biliary excretion mechanisms for irinotecan, CPT-11, and its metabolites in rats. Cancer Research, 57(10), 1934-1938.
Feun, L., Savaraj, N. (2008). Topoisomerase I inhibitors for the treatment of brain tumors. Expert Rev Anticancer Ther., 8, 707-716.
Kan, S. F., Huang, W. J., Lin, L. C., & Wang, P. S. (2004). Inhibitory effects of evodiamine on the growth of human prostate cancer cell line LNCaP. International journal of cancer, 110, 641-651.
Ko, H. C., Wang, Y. H., Liou, K. T., Chen, C. M., Chen, C. H., Wang, W. Y., Chang, S., Hou, Y. C., Chen K. T., Chen, C. F., Shen, Y. C. (2007). Anti-inflammatory effects and mechanisms of the ethanol extract of Evodia rutaecarpa and its bioactive components on neutrophils and microglial cells. Eur J Pharmacol., 555, 211-217.
Liu, L. F. (1989). DNA topoisomerase poisons as antitumor drugs. Annual review of biochemistry, 58, 351-375.
Ogasawara, M., Matsunaga, T., Takahashi, S., Saiki, I., & Suzuki, H. (2002). Anti-invasive and metastatic activities of evodiamine. Biological & pharmaceutical bulletin, 25, 1491-1493.
Sadaie, M. R., Mayner, R., Doniger, J. (2004). A novel approach to develop anti-HIV drugs: adapting non-nucleoside anticancer chemotherapeutics. Antiviral Res., 61, 1-18.
Sekiguchi, J., Cheng, C., & Shuman, S. (1997). Kinetic analysis of DNA and RNA strand transfer reactions catalyzed by vaccinia topoisomerase. The Journal of biological chemistry, 272, 15721-15728.
Song, J., Parker, L., Hormozi, L., Tanouye, M. A. (2008) DNA topoisomerase I inhibitors ameliorate seizure-like behaviors and paralysis in a Drosophila model of epilepsy. Neuroscience. (In Press)
Staker, B. L., Hjerrild, K., Feese, M. D., Behnke, C. A., Burgin, A. B. Jr, Stewart, L. (2002). The mechanism of topoisomerase I poisoning by a camptothecin analog. Proc Natl Acad Sci USA. 99, 15387-15392.
Teicher, B. A. (2008). Next generation topoisomerase I inhibitors: Rationale and biomarker strategies. Biochemical pharmacology, 75, 1262-1271.
Verdrengh, M., Tarkowski, A. (2003). Impact of topoisomerase II inhibition on cytokine and chemokine production. Inflamm Res., 52, 148-153.
Wang, T., Wang, Y., Kontani, Y., Kobayashi, Y., Sato, Y., Mori, N., et al. (2008). Evodiamine improves diet-induced obesity in a uncoupling protein-1-independent manner: involvement of antiadipogenic mechanism and extracellularly regulated kinase/mitogen-activated protein kinase signaling. Endocrinology, 149, 358-366.
Wethington, S. L., Wright, J. D., Herzog, T. J., (2008). Key role of topoisomerase I inhibitors in the treatment of recurrent and refractory epithelial ovarian carcinoma. Expert Rev Anticancer Ther., 8, 819-831.
Yoshinari, T., Yamada, A., Uemura, D., Nomura, K., Arakawa, H., Kojiri, K., et al. (1993). Induction of topoisomerase I-mediated DNA cleavage by a new indolocarbazole, ED-110. Cancer research, 53, 490-494.
Zhou, Y., Li, S. H., Jiang, R. W., Cai, M., Liu, X., Ding, L. S., Xu, H. X., But, P. P., Shaw, P. C. (2006). Quantitative analyses of indoloquinazoline alkaloids in Fructus Evodiae by high-performance liquid chromatography with atmospheric pressure chemical ionization tandem mass spectrometry. Rapid Commun Mass Spectrom., 20, 3111-3118.

### DETAILED DESCRIPTION OF THE PREFERRED EMBODIMENTS

The present invention discloses that a drug target of EVO is TopoI, thereby providing a platform of exploiting a new anticancer drug. By a stereo binding property of TopoI, evodiamine, rutaecarpine or dehydroevodiamine can be used as a pilot compound to design a new drug capable of combining DNA topoisomerase more tightly, and that will be a great contribution in medical development. The activity analysis can be proceed using a computer molecular dynamics simulation technology (i.e. molecular modeling) (Staker, et al. 2002), a supercoiled DNA relaxation activity test or a binding activity test of a topoisomerase I and DNA complex in vivo. Wherein TopoI may be prepared from a virus, a eukaryotic cell, a recombinant DNA expression in a host.

### Examples

Example 1 relates to cell growth inhibition of EVO according to the present invention. Breast cancer MCF-7 cells are cultured in Dulbecco's modified Eagle medium (DMEM) supplemented with 10% heat-inactivated fetal bovine serum (FBS), and 100 μg/ml penicillin-streptomycin. Conditions were maintained in a humidified 95% air/5% CO₂ incubator at 37° C. The MTT [3-(4,5-dimethylthiazol-2-yl)-2,5-diphenyl tetrazolium bromide] assay was described previously to test the cytotoxicity of reagents and cell viability (Lin, Tsai, Chen, Chang, Lee et al., 2008). Cells (5000 cells/well) are grown on a 96-well plate supplemented with DMEM medium (with 1% FBS) for 24 hours. Then, cells are treated with CPT or EVO (0˜0.30 μM), and the viability is determined by the reduction of MTT. An MTT stock solution (5 mg of MTT/ml of PBS) is added to the growing cultures (at a final concentration of 0.5 mg/ml). The OD is measured with a spectrophotometer at 560 nm. A blank with DMSO alone is measured and subtracted from all values.

The cytotoxic abilities of EVO and CPT are tested against human breast MCF-7 carcinoma cells, which express high levels of TopoI. CPT is used as a reference drug. Both compounds display efficient cytotoxicity after 24 h of drug exposure. The IC₅₀ values are 3.23 μM for CPT and 6.02 μM for EVO. Consequently, EVO is slightly less cytotoxic than CPT against breast MCF-7 carcinoma cells.

Example 2 relates to an effect of EVO inhibiting supercoiled plasmid DNA relaxation catalyzed by TopoI from a vaccinia virus according to the present invention. DNA TopoI from the vaccinia virus (EPICENTRE Biotechnologies, Madison, Wis.) is a type I eukaryotic topoisomerase that catalyzes the breakage and formation of phosphodiester bonds in a single strand of a duplex DNA molecule. The enzyme cleaves at the 3′ side of a specific target sequence [5′(C/T)CCTT⁻] of DNA and relegates the original phosphodiester bond that relaxes DNA, resulting in the conversion of supercoiled DNA to relaxed closed circular DNA. The inhibitory effect of CPT and EVO on supercoiled DNA strand breakage caused by TopoI is evaluated. pcDNA3 plasmid DNA (200 ng) is incubated at 37° C. for 30 minutes in a reaction solution (50 mM Tris-acetate, 100 mM NaCl, 2.5 mM MgCl₂, and 0.1 mM EDTA; pH 7.5) in the presence or absence of 0˜3.0 μM inhibitor in a final volume of 20 μl (Sekiguchi, Cheng & Shuman, 1997). The conversion of the covalently closed circular double-stranded supercoiled DNA to a relaxed form is used to evaluate DNA strand breakage induced by TopoI. Samples are loaded onto a 1% agarose gel, and electrophoresis is performed in TAE buffer (40 mM Tris-acetate and 1 mM EDTA), then the gel is photographed under transmitted ultraviolet light.

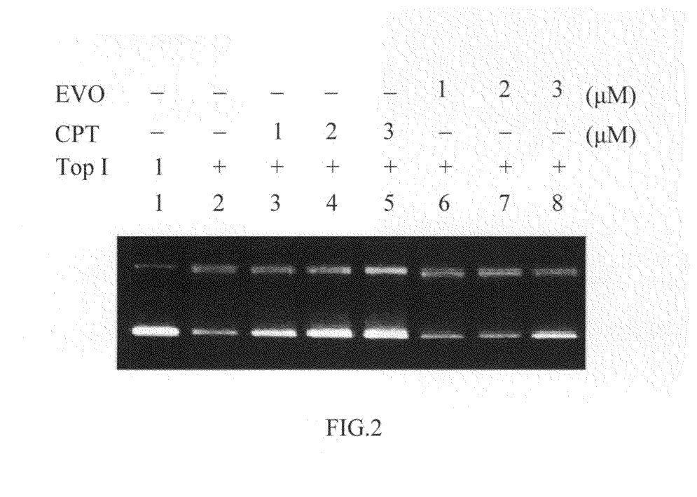

TopoI is known to relax the supercoiled plasmid DNA to an open circular form in vitro and in vivo. CPT and EVO inhibition of supercoiled DNA relaxation in vitro was evaluated. Vaccinia TopoI's induction of supercoiled pcDNA3 plasmid relaxation is employed as the assay system, and the results are shown in FIG. 2. Supercoiled DNA migrated faster on the agarose gel than the relaxed circular DNA as shown in the control (FIG. 2, lanes 1 and 2). CPT treatments retain a greater amount of uncatalytic supercoiled DNA in a dose-dependent manner (lanes 3-5, 1-3 μM). EVO also displays inhibitory activity on TopoI catalytic relaxation in a dose-dependent manner (lanes 6-8, 1-3 μM). These results suggest that EVO is able to inhibit supercoiled plasmid DNA relaxation catalyzed by TopoI. EVO reveals similar inhibition activities toward purified recombinant eukaryotic TopoI and nuclear preparation of MCF-7 cells.

The activity of supercoiled DNA relaxation catalyzed by TopoI prepared from recombinant DNA is also inhibited by EVO. Inhibitory activity of rutaecarpine on TopoI catalytic supercoiled plasmid relaxation is substantially equivalent to those of CPT and EVO.

Example 3 relates to detection of the levels of free-form TopoI from breast cancer cells reduced by EVO according to the present invention. Provided that EVO is able to fix TopoI on DNA, the level of free-form TopoI should be decreased. Monolayers that are breast cancer MCF-7 cells of 50%—80% confluent are treated with drugs for a short time, and then cellular proteins are extracted from the MCF-7 cells. After that, the cellular proteins are separated electrophoretically in a 7.5% SDS polyacrylamide gel and electro-transferred to a polyvinylidene difluoride (PVDF) membrane. The membrane is incubated with the primary antibody (rabbit anti-human topoisomerase I antisera) at room temperature for 2 hours, and then incubated with a horseradish peroxidase-conjugated secondary immunoglobulin G (IgG) antibody. The immunoreactive bands are visualized with enhanced chemiluminescent reagents and photographed using a gel documentation system.

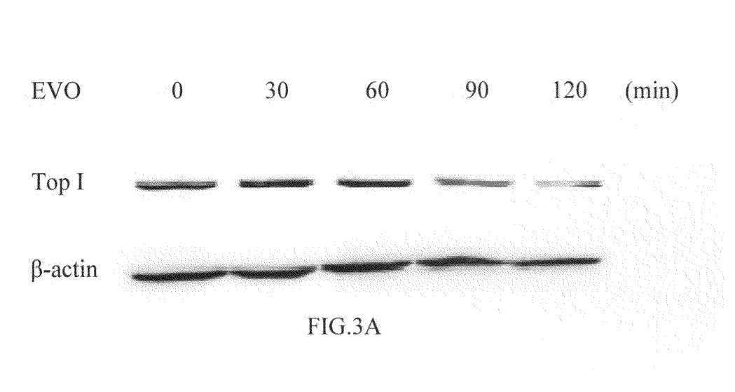

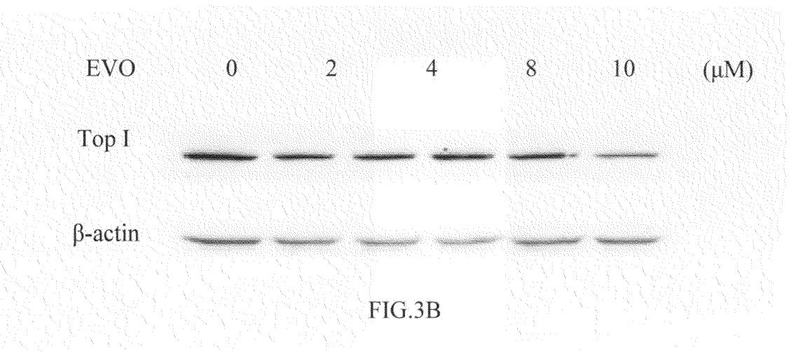

A depletion assay for free-form TopoI is performed to confirm the effect of EVO on DNA topological catalysis of TopoI. This assay is based on the ability of CPT to trap TopoI and DNA to form a CPT-TopoI-DNA triple complex, to see if EVO inhibits TopoI activity through a similar mechanism. The degree to which depletion occurs depends on the extent to which TopoI binds DNA, which is a function of EVO sensitivity. MCF-7 cells maintained in normal conditions express a detectable level of TopoI protein. MCF-7 cells are treated with EVO for 0˜120 minutes. The protein levels of TopoI are examined by immunoblotting. The levels of TopoI in EVO treatments (10 μM) are depleted in a time-dependent manner, in that the protein level decreased to <20% with 120-minute treatment in comparison to the untreated control (FIG. 3A). Depletion of TopoI protein by EVO is also detected in a concentration-dependent manner after 1 hour of treatment. The relative level of DNA-unlinked TopoI protein after treatment with 0˜10 μM EVO decreased to <40% versus the control (FIG. 3B). β-Actin with constant expression is used as the internal control.

Example 4 relates to using a KCl/SDS precipitation assay to show TopoI-DNA complex trapping activity of EVO according to the present invention. The formation of a cleavable complex in intact cells is quantified by a KCl-sodium dodecylsulfate (SDS) precipitation technique, a modified procedure described previously by Yoshinari et al. (1993). Cellular DNA is labeled by adding ³H-thymidine to the medium to a final concentration of 10 μCi/ml. After an overnight incubation, cells are plated to a density of 1×10⁵ cells/well in a 24-well plate for another overnight incubation, and treated with various concentrations of EVO (0˜30 μM) for 60 minutes. The medium is removed from each well, and cells are washed with phosphate-buffered saline (PBS) and lysed with 1 ml of prewarmed (65° C.) lysis solution (1.25% SDS, 5 mM EDTA, 0.4 mg/ml salmon sperm DNA). Lysate is sheared using a 21-gauge needle. The samples as background control are treated by the above procedure but with proteinase K (400 μg/ml) in the lysis buffer, and are incubated at 50° C. for 2 hour. KCl (325 mM), at 250 μl, is added to each sample, vortexed vigorously, cooled on ice for 10 minutes, and centrifuged at 2500 rpm for 10 minutes at 4° C. The pellet is washed twice in 1 ml of wash solution (10 mM Tris-HCl, 100 mM KCl, 1 mM EDTA, and 0.1 mg/ml salmon sperm DNA) and incubated at 65° C. for 10 minutes, cooled on ice, then centrifuged at 2500 rpm for 10 minutes. The pellet is resuspended in 400 μl prewarmed H₂O (65° C.) and combined with 4 ml scintillation liquid, and the radioactivity counts are determined using a liquid scintillation counter.

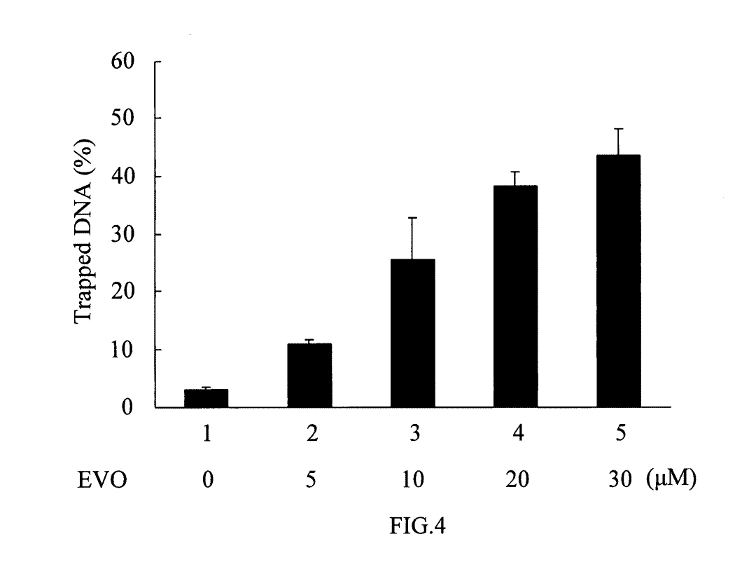

The TopoI-DNA complex can be precipitated using the KCl/SDS precipitation method. The amount of precipitated DNA reflects the EVO-trapping activity. ³H-thymidine-labeled cells are treated with EVO (0, 5, 10, 20, and 30 μM, respectively) for 60 minutes, and in vivo the KCl-SDS precipitation assay is performed. Only 3.1% of the ³H-thymidine-labeled DNA is trapped in a covalent complex with TopoI in 0 μM-treated MCF-7 cells. DNA trapped by EVO increased to 43.6% with 30 μM treatment (FIG. 4). The result indicates that the ability of EVO to cause the formation of the TopoI-DNA complex increases with EVO treatment in a dose-dependent manner.

Example 5 relates to a molecular modeling according to the present invention. The molecular modeling is to set a binding site of a new drug with 3-D structure to be a domain of molecular docking as a screening range by using molecular modeling software. A 3-D structure of the covalent TopoI-DNA complex stabilized by CPT was solved (Staker, et al. 2002), thereby easily proceeding a virtual screening by binding. After finishing the dock screening, a combination mode with a high score is detected according to a binding way of a compound and an enzyme, such as van der Waals force or electrostatic interaction, using rigid-body docking computer software. The binding way having a higher score is a possible binding way of an inhibitor and an activity center of the enzyme on a ligand. Referring to the result of the molecular modeling, a binding activity of a new drug to inhibit the covalent TopoI-DNA complex is analyzed (Chang, et al. 2007).

While particular embodiments of the present invention have been shown and described, it will be obvious to those skilled in the art that, based upon the teachings herein, changes and modifications may be made without departing from this invention and its broader aspects. Therefore, the appended claims are intended to encompass within their scope of all such changes and modifications as are within the true spirit and scope of the exemplary embodiments of the present invention.

What is claimed is:

1. A pharmaceutical composition for inhibiting topoisomerase I, the pharmaceutical composition comprising evodiamine or derivatives thereof, the derivatives comprising one of evodiamine, rutaecarpine and dehydroevodiamine in combination of a pharmaceutically acceptable salt, solvate, or physiological function derivative thereof.

2. The pharmaceutical composition according to claim 1, wherein the pharmaceutical composition inhibits a proliferating cell having topoisomerase I activity.

3. The pharmaceutical composition according to claim 2, wherein the proliferating cell is selected from a group consisting of a eukaryotic cell, virus and bacterium.

4. The pharmaceutical composition according to claim 1, wherein the topoisomerase I is provided from a virus.

5. The pharmaceutical composition according to claim 1, wherein the topoisomerase I is provided from a eukaryotic cell.

6. The pharmaceutical composition according to claim 1, wherein the topoisomerase I is provided by a recombinant DNA expression in a host.

7. A method for exploiting a drug, characterized by using evodiamine, rutaecarpine or dehydroevodiamine as a pilot compound to provide a derivative comprising a topoisomerase I inhibitory activity.

8. The method for exploiting a drug according to claim 7, wherein the method is proceeded using a computer molecular dynamics simulation technology.

9. The method for exploiting a drug according to claim 7, wherein the method is proceeded using a test in supercoiled DNA relaxation activity.

10. The method for exploiting a drug according to claim 7, wherein the method is proceeded using a test in binding activity of a topoisomerase I and DNA complex.

11. The method for exploiting a drug according to claim 7, wherein the topoisomerase I is provided from a virus.

12. The method for exploiting a drug according to claim 7, wherein the topoisomerase I is provided from a eukaryotic cell.

13. The method for exploiting a drug according to claim 7, wherein the topoisomerase I is provided by a recombinant DNA expression in a host.
